(19) United States
(12) Patent Application Publication
(10) Pub. No.: US 2017/0172895 A1
(43) Pub. Date: Jun. 22, 2017

(54) Topical Anhydrous Fill Formulation For Ultraviolet Filters

(71) Applicant: R.P. SCHERER TECHNOLOGIES, LLC, Las Vegas, NV (US)

(72) Inventors: Ilaria Marigliano, Rome (IT); Laura Pironi, Rome (IT)

(73) Assignee: R.P. Scherer Technologies, LLC, Las Vegas, NV (US)

(21) Appl. No.: 15/115,390

(22) PCT Filed: Feb. 18, 2015
(86) PCT No.: PCT/US15/16267

§ 371 Date: Jul. 29, 2016

Related U.S. Application Data
(60) Provisional application No. 61/941,200, filed on Feb. 18, 2014.

(51) Int. Cl. A61K 8/891; A61K 8/92; A61Q 17/04; A61K 8/89

(52) U.S. Cl. CPC A61K 8/891; A61K 8/89; A61K 8/92; A61Q 17/04

(57) ABSTRACT

An anhydrous base composition including a branched hydrocarbon base oil having 9-16 carbon atoms and a viscosity adjusting agent suitable for use in the formulation of a cosmetic composition and which is compatible with a variety of capsule technologies. Also described is a storage stable anhydrous fill formulation compatible with a variety of different capsule technologies and which includes an anhydrous base composition including a base oil and a viscosity adjusting agent, as well as one or more UV components such as UV filters and, optionally, one or more UV defense boosters. Soft capsule formulations containing UV filters are also disclosed. The soft capsule formulations may include a soft capsule shell and an anhydrous fill material within the shell, which, when dispensed, provides a film including one or more sun protection ingredients.

### BACKGROUND OF THE INVENTION

1. Field of the Invention

The invention relates to fill formulations providing a stable anhydrous vehicle for ultraviolet (UV) filters and optionally one or more UV defense boosters. More specifically, the fill formulation provides the ability to include sun protection in skin care formulations that are compatible with capsules, pearls and drops unit dose technologies.

2. Description of the Related Technology

There are a lot of theories on aging and some established ones consider not only the chronologic role, which is related to genetic, structural factors as well as to a series of intrinsic phenomena (Chrono-aging), but also the role of photo induced free radicals, which accelerate and anticipate skin aging (Photo-aging). Photo-aging depends primarily on the degree of sun exposure and skin pigment (photo-type).

It is well known that it is important to protect your skin from the sun every day. Whether spending a day at the beach or running errands, sun protection is essential. You should apply sunscreen every day to all skin that is not covered by clothing. Increasingly, a variety of skincare products have an SPF built in for day to day protection.

It would be of value to have soft gelatin non gelatin based (also known as Vegicaps® capsules or OptiShell™ capsules) capsules encapsulating topical formulations containing UV filters.

Sun protection in the form of a variety of different products is currently in demand in the marketplace. However, such sun protection must deliver a minimum Sun Protection Factor (SPF), typically at least SPF 15 is required. However, many difficulties have been encountered in the formulation of topical products that are compatible with capsule shell technologies such as gelatin based capsules and Vegicaps® or OptiShell™ capsules and which meet actual market requirements for products containing UV filters and UV defense boosters.

One challenge is that a drop in viscosity is observed, particularly in anhydrous formulations, when a UV filter is added to the formulation. The drop in viscosity has a negative effect on the texture and skin feeling of the product and an impact on the film forming property of the formulation when applied to the skin. Also, the viscosity of the product influences the physical stability of the formulation, as well as the ability to reproducably encapsulate the formulation. The film-forming and textural properties may be important for topical care products with a Sun Protection Factor and skincare products. For example, the film-forming property of the formulation affects the Sun Protection Factor that is provided by the product when applied to the skin. The textural or skin feel properties may be important for consumer acceptance of the product. Furthermore, it is desirable that suitable formulations should allow for inclusion of other skin care actives in the formulations.

The use of soft gelatin and Vegicaps® capsules to deliver topical formulations including UV ingredients would provide an attractive, convenient single use option for dispensing fill compositions containing UV ingredients potentially providing an SPF rating. Typically, these capsules contain a unit dose of from about 0.09 ml to about 4.5 ml of fill material and have a “twist-off” or other removable feature at one end for dispensing the fill material. Such Soft gelatin and Vegicaps® capsules can be prepared by methods well known for the preparation of softgels for oral dosage formulations, i.e. by encapsulating the fill material between two sheets of gelatin as it passes between a pair of die rolls having surface cavities shaped to form the desired shape of the resulting softgel.

It is also desirable to provide formulations that are compatible with standard and other encapsulation technologies and capsule forms including Jintan™ and liquid filled two-piece hard shell capsule technologies.

The present invention provides formulations allowing inclusion of UV ingredients that support a SPF, and provides compatibility with different encapsulation technologies and capsule forms.

### SUMMARY OF THE INVENTION

In one aspect, the present invention provides an anhydrous base composition comprising a branched hydrocarbon base oil having 9-16 carbon atoms and a viscosity adjusting agent.

In a second aspect, the present invention provides a storage stable anhydrous fill formulation compatible with a variety of different capsule technologies and which comprises an anhydrous base composition including a base oil and a viscosity adjusting agent, as well as one or more UV components such as UV filters and optionally one or more UV defense boosters.

In a third aspect, this invention provides soft capsule formulations containing UV filters, comprising a soft gelatin capsule shell and an anhydrous fill material within the shell, which, when dispensed, provides a film including one or more sun protection ingredients. The fill material may be a fill material in accordance with the invention and may include thickened hydrocarbon oils, UV filters and optionally one or more UV defense boosters.

### BRIEF DESCRIPTION OF THE DRAWINGS

FIG. 1 shows the results of a subjective assessment of the short term-effects of the product of Example 1.

### DETAILED DESCRIPTION OF THE PREFERRED EMBODIMENT(S)

For illustrative purposes, the principles of the present disclosure are described by referencing various exemplary embodiments. Although certain embodiments are specifically described herein, one of ordinary skill in the art will readily recognize that the same principles are equally applicable to, and can be employed in other devices and methods. Before explaining the disclosed embodiments of the present disclosure in detail, it is to be understood that the disclosure is not limited in its application to the details of any particular embodiment shown. Additionally, the terminology used herein is for the purpose of description and not of limitation. Furthermore, although certain methods are described with reference to steps that are presented herein in a certain order, in many instances, these steps may be performed in any order as may be appreciated by one skilled in the art; the novel method is therefore not limited to the particular arrangement of steps disclosed herein.

It must be noted that as used herein and in the appended claims, the singular forms “a”, “an”, and “the” include plural references unless the context clearly dictates otherwise. Furthermore, the terms “a” (or “an”), “one or more” and “at least one” can be used interchangeably herein. The terms “comprising”, “including”, “having” and “constructed from” can also be used interchangeably.

As used herein, “capsule” refers to a two piece hard shell or a soft gelatin and/or non-gelatin capsule manufactured utilising a rotary die machine.

As used herein, “unit dose” refers to a dose of from about 0.09 ml to 4.5 ml.

In one embodiment, the invention relates to a base for a fill formulation including a thickened hydrocarbon component and a UV component. The UV component may include one or more UV filters and optionally one or more UV defense boosters. This base can be used for a wide range of formulations including formulations containing skin care components and formulations designed for use a various types of antiaging products.

In another embodiment, the invention relates to a fill formulation adapted for single-use application using a single-use container. The fill formulation contains one or more UV ingredients. The fill formulation is storage stable, capable of film formation upon application and is compatible with a variety of different single-use container technologies including softgel capsules and hard shell capsules.

In another embodiment, the invention relates to a softgel capsule formulation containing one or more UV ingredients in the fill material. Formulations according to this invention are stable and provide cosmetically acceptable topical composition and do not destabilize softgel capsules including, but not limited to, Softgel or Vegicaps® capsules, as well as being compatible with pearls and drops, Thus, the present invention provides attractive topical formulations for UV filters or sunscreens, optionally including one or more UV defense boosters in convenient single-use containers. The single dose formulations of the present invention can also be used in other anhydrous fill/oily fill compatible systems, such as, for example, two-piece hard shell capsules, Jintan capsules, animal and non-gelatin based capsule shell technologies and Vegicaps® capsule formulations.

The compatibility of the fill formulation with both UV ingredients and capsule technologies leads to the ability to provide a composition in unit dose form that is capable of forming a homogenous film on the skin. As a result, the composition may be used as a vehicle for a sun protection factor (SPF), as well as being able to deliver other desirable products features for the skin. The texture of the formulation in the invention permits inclusion of UV ingredients including at least UV filters and optionally one or more UV defense boosters which, in turn, provide a SPF usable in a wide range of skin care formulations including those delivered by a unit dose delivery form such as capsules.

The base composition of the invention may include a variety of ingredients. In one embodiment, the base composition includes a thickened hydrocarbon base adapted for supporting one or more UV filters, and optionally one or more UV defense boosters. The thickened hydrocarbon base can be used to form a fill composition that is in the form of an anhydrous topical product.

The base composition may contain one or more light partially volatile oils. Suitable oils are hydrocarbons containing only carbon and hydrogen. In one embodiment, the hydrocarbon oil may be a branched chain aliphatic hydrocarbon having 9 to 16 carbon atoms. One example of a hydrocarbon oil is isododecane.

Another class of volatile oils useful for the present invention include low viscosity linear siloxanes such as, for example, disiloxane, trisiloxane and dimethicone. The volatile oils may, in some embodiments, function as a solvent for the viscosity control agents described below.

The viscosity of the hydrocarbon oil is adjusted using one or more suitable viscosity control agents. In certain embodiments, the hydrocarbon oil component is thickened, or gelled, to increase its viscosity using one or more viscosity control agents that are thickening agents either taken alone or in combination. Suitable viscosity control agents include, for example, silica, fumed silica and its reaction products such a silica silylate, silica dimethyl silylate and silica dimethicone silylate, and other anhydrous viscosity control agents. For example, viscosity control agents obtained by the reaction of a quaternary ammonium salt and hectorite or bentonite, such as disteardimonium hectorite, stearalkonium bentonite and stearalkonium hectorite. Hectorite is one of the montmorillonite minerals that are the principal constituents of bentonite clay. Bentonite is a native hydrated colloidal aluminum silicate clay. Other suitable viscosity control agents include, for example, carbonates such as propylene carbonate and dicapryl carbonate, cross-linked siloxane polymers, polydimethylsiloxanes, and synthetic waxes such as a polyethylene polymer, which is predominantly made up of polymer units having at least 30 or more carbon atoms. The polyethylenes may have a melting point range of 50°−120° C.

In some embodiments, it may be desirable to employ a combination of two or more viscosity control agents to provide a balance between viscosity, stability, skin feel, film-forming ability and the ability to encapsulate the material. One class of viscosity modifiers useful for this purpose are silicone elastomers. The silicone elastomers may be used in combination with one or more other classes of viscosity modifying agents. For example, a combination of one or more silicone elastomers with one or more synthetic waxes may be employed. Other combinations include silicone elastomers with one or more clay-based viscosity control agents, or combinations of silicone elastomers, synthetic waxes and clay-based viscosity-control agents or combinations of synthetic waxes and clay-based viscosity control agents. Each of the foregoing combinations may be further combined with one or more silica-based viscosity control agents.

Silicone elastomer viscosity control agents include, for example, cross-linked siloxane polymers and polydimethylsiloxanes. More specific examples include INCI polysilicone-11, a crosslinked dimethyl siloxane formed by the reaction of bis-vinyldimethicone and hydrogen dimethicone in the presence of a solvent. Bis-vinyldimethicone is a derivative of dimethicone wherein one methyl group at each end of the siloxane chain has been replaced with a vinyl group. Hydrogen dimethicone is a derivative of dimethicone wherein some of the methyl groups have been replaced with a hydrogen atom. INCI stands for International Nomenclature for Cosmetic Ingredients.

Another suitable cross-linked siloxane polymer is INCI vinyldimethyl/trimethylsiloxysilicate/dimethicone crosspolymer which is formed by crosslinking hydrogen dimethicone with vinyldimethyl/trimethylsiloxysilicate.

Another suitable cross-linked siloxane polymer is INCI dimethicone/vinyl dimethicone crosspolymer which is a crosslinked dimethyl siloxane polymer formed by the reaction of hydrogen dimethicone and vinyl dimethicone. Vinyl dimethicone is a derivative of dimethicone where some of the methyl groups have been replaced with vinyl groups. The vinyl groups can be present at the ends of the siloxane chain or pendant to the siloxane chain.

Another suitable cross-linked siloxane polymer is INCI dimethicone crosspolymer, which is a polymer of dimethicone crosslinked with an alkyl group and is available as Dow Corning EL 8040ID. Mixtures of two or more of these silicone elastomers may be used alone or in combination with other viscosity control agents.

The amount of silicone elastomer viscosity control agent employed in the fill composition may be from about 4.0 to about 16.0 wt %, and, more preferably from about 5 to about 14 wt %.

Synthetic wax viscosity control agents, include, for example, polyethylene polymers, which are predominantly made up of polymer units having at least 30 or more carbon atoms. The preferred polythylene wax may include low levels such as between 0.1 and 10%, or, more preferably between 0.5-5% of polymer units having 26 or more carbon atoms with a melting point range of 60°-80° C. The polyethylenes are preferred since they thicken, are film-forming and can improve the skin feel of the product. Mixtures of two or more of these synthetic wax materials may be used alone or in combination with other viscosity control agents. The amount of synthetic wax viscosity control agent employed in the fill composition may be from about 0.1 to about 7 wt. %, and, more preferably from about 0.5 to about 5 wt %.

Clay-based viscosity control agents include, for example, viscosity control agents obtained by the reaction of a quaternary ammonium salt and hectorite or bentonite, such as disteardimonium hectorite, stearalkonium bentonite and stearalkonium hectorite. Hectorite is one of the montmorillonite minerals that are the principal constituents of bentonite clay. Bentonite is a native hydrated colloidal aluminum silicate clay. Mixtures of two or more of these materials may be used alone or in combination with other viscosity control agents. The amount of the clay-based viscosity control agent employed in the fill composition may be from about 0.1 to about 2 wt %, and, more preferably from about 0.15 to about 1 wt %.

Silica-based viscosity control agents include, for example, silica, fumed silica and its reaction products such a silica silylate, silica dimethyl silylate and silica dimethicone silylate, and other anhydrous viscosity control agents. Mixtures of two or more of these materials may be used alone or in combination with other viscosity control agents. The amount of the silica-based viscosity control agent employed in the fill composition may be from about 0.01 to about 5 wt %, and, more preferably from about 0.02 to about 3.5 wt %.

One particularly suitable base oil composition is a thickened hydrocarbon oil composition, such as isododecane thickened with a cross-linked siloxane polymer such as INCI polysilicone-11, INCI vinyldimethyl/trimethylsiloxysilicate/dimethicone crosspolymer, INCI dimethicone/vinyl dimethicone crosspolymer and INCI dimethicone crosspolymer.

The invention also relates to fill formulations useful as cosmetic agents, skincare products, over the counter (OTC) topical products, topical medical products, pharmaceutical products as well as other types of topical products. The fill formulations of the present invention may comprise the base composition described above and a UV component such as a UV filter, and optionally one or more UV defense boosters. The fill formulations may optionally further comprise one or more additional UV filters. Exemplary fill formulations in accordance with the invention may include, for example, the ingredients set forth in Table 1.

TABLE 1

| | | Preferrred | More Preferrred |
|---|---|---|---|
| Component | Wt. % | Wt % | Wt. % |
| Thickening agent(s) and/or | 5-70 | 8-40 | 10-20 |
| Viscosity control agent(s) | | | |
| Hydrocarbon oil(s) | 20-70 | 30-65 | 40-60 |
| UV filter(s) | 7-30 | 8-20 | 8-15 |
| UV stabilizer(s) | 0.0-10 | 0.5-10 | 3-8 |
| Skin conditioning agent(s) | 0-20 | 5-20 | 10-20 |

A UV filter is an ingredient that absorbs, reflects, or scatters radiation in the UV wavelength range, specifically at wavelengths of from 290 to 400 nanometers. The fill composition of the present invention may include one or more UV filters. One type of UV filter that may be employed in the present invention are UV filter substances based on triazine derivatives. One class of UV filters that may be used in the present invention include alkyl methoxy cinnamates such as ethyl hexyl methoxy cinnamate, octyl methoxy cinnamate, and isoamyl methoxy cinnamate. Other UV filters that may be used in the present invention include oxybenzone, homosalate, octocrylene, octyl salicylate, avobenzone, titanium dioxide, and zinc oxide. Diethylamino hydroxybenzoyl hexyl benzoate may also be used as a UV filter substance.

UV defense boosters are ingredients such as ingredients that improve formulation efficiency, photostabilizers, film-forming agents, antioxidants, and ingredients which improve solvency of the UV filters. The fill formulations may optionally include one or more of each of these UV boosters as well as mixtures of these UV boosters.

Photostabilizers are employed in cosmetics to protect the product from chemical or physical deterioration induced by light. In many products, a combination of UV filters and photostabilizers is utilized to protect the skin or hair from ultraviolet light. The fill compositions of the present invention may include photostabilizers or a combination of one or more UV filters and one or more photostabilizers. Suitable photostabilizers may include, for example, diethylamino hydroxybenzoyl hexyl benzoate, ethylhexyl methoxycrylene and diethylhexyl syringylidene malonate.

One particularly useful combination of a UV filter and a photostabilizer is ethylhexyl methoxycinnamate in combination with diethylamino hydroxybenzoyl hexyl benzoate. This combination, alone or in combination with other photostabilizers can provide an SPF of 15 or an SPF of more than 20, as measured by in vivo determination using the method of ISO24444 and a UVA protection factor (PF) of 7.2, as measured by in vitro determination using the method of ISO24443.

The final viscosity range of the fill composition is from 10,000 cP (centipoise) to 60,000 cP, or from 20,000 cP to 50,000 cP.

The fill formulation may include other optional ingredients such as, for example, compounds that have a photo-protective effect, skin brighteners, skin conditioners to improve appearance, restore suppleness and protect the skin, skin smoothing ingredients, skin soothing agents, moisturizers, melanin inhibitors, free radical quenchers, anti-inflammatories and mixtures thereof. These additional materials may be employed in conventional amounts known to skilled formulators of products employing these ingredients.

The fill formulations of the present invention may be employed for daily use in the form of a skin care product that helps to protect the skin from sun damage and may, optionally, also help to prevent premature skin-aging. These products may be conveniently offered in softgel or Vegicaps® capsules in unit-dosages, for on-the-go application. The fill formulation is formulated such that when it is dispensed, it forms a film on the skin that is substantially homogeneous since this can improve the SPF of the product upon application.

Thus, the formulations of the present invention can be employed to provide sun protection, e.g. to provide an SPF of at least 15 or an SPF of at least 20. One or more UV filters and optionally one or more UV defense boosters can be employed to provide SPF 15 or 20, if desired.

Anti-ageing, skincare, pharmaceutical or cosmetic formulations in accordance with the present invention can be formulated to smooth wrinkles, particularly in the short term, to brighten skin, to help reduce and prevent melanic dark spots, and/or to provide free-radical protection. In one embodiment, the invention provides a product for daily use that is formulated to combine a short term wrinkle smoothing effect and sun protection with a long term skin brightening effect.

In a further aspect, the present invention relates to a capsule containing a fill formulation as described above. The capsules may be, for example, softgel or Vegicaps® capsules. In other embodiments, the capsule may be a hard shell capsule. In some embodiments, the capsules comprise gelatin and in other embodiments the capsules do not employ gelatin in the capsule composition. In one embodiment, the capsule is sized to provide a unit-dosage of the fill formulation.

Number ranges given in the specification, such as size ranges and the like, should be considered approximate, unless specifically stated. Ingredient names are taken from the wINCI Web Based International Cosmetic Ingredient Dictionary & Handbook and the CosIng cosmetic ingredients database.

### Example 1

The following example is illustrative, but not limiting, of the methods and compositions of the present disclosure. Other suitable modifications and adaptations of the variety of conditions and parameters normally encountered in the field, and which are obvious to those skilled in the art, are within the scope of the disclosure.

The formulation of Table 2 was employed to provide a sun protection factor >/=15 for a daily skin care product. The primary ingredients of the formulation are set forth in Table 2 below with the amounts given in parts per hundred by weight, based on the total weight of the formulation. The formulation included isododecane as the base oil and polyethylene, silica, dimethicone crosspolymer, stearalkonium hectorite, propylene carbonate and dicapryl carbonate as viscosity control agents. The formulation included an ethylhexyl methoxycinnamate UV filter as well as diethylamino hydroxybenzoyl hexyl benzoate, ethylhexyl methoxycrylene and diethylhexyl syringylidenemalonate for the purpose of protecting the skin from sun damage and to help prevent photo-ageing.

TABLE 2

| Ingredient | pph |
|---|---|
| ISODODECANE | 50.2600 |
| DIMETHICONE CROSSPOLYMER | 7.0400 |
| ETHYLHEXYL METHOXYCINNAMATE | 7.0000 |
| NEOPENTYL GLYCOL DIHEPTANOATE | 5.7000 |
| SILICA | 4.0000 |
| ETHYLHEXYL METHOXYCRYLENE | 4.0000 |
| POLYETHYLENE | 4.0000 |
| C12-15 ALKYL BENZOATE | 4.0000 |
| DIETHYLAMINO HYDROXYBENZOYL HEXYL | 3.5000 |
| BENZOATE | |
| CAPRYLIC/CAPRIC TRIGLYCERIDE | 3.1946 |
| DICAPRYLYL CARBONATE | 2.6100 |
| DIETHYLHEXYL SYRINGYLIDENEMALONATE | 1.8000 |
| ETHYLHEXYL PALMITATE | 0.9530 |
| STEARALKONIUM HECTORITE | 0.3000 |
| PROPYLENE CARBONATE | 0.0900 |
| SILICA DIMETHYL SILYLATE | 0.0250 |

The composition of Table 2 was tested for UV protection factor and the results are given in Table 3 below.

TABLE 3

| | |
|---|---|
| SPF - UVB Protection | 24.2 |
| In vivo determination- | |
| ISO24444 | |
| UVA PF (Protection Factor) | 7.2 |
| In vitro determination - | |
| ISO24443 | |

In vitro Critical wavelength evaluation with pre-irradiation COLIPA 2011 method: 372 nm

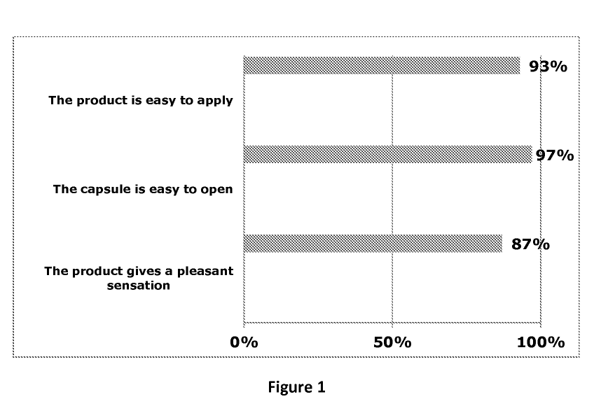

A subjective assessment of the product of Table 2 was carried out by providing the product to 30 volunteer test subjects ages 18 to 55. One daily application of the product on the fact was carried out by the test subjects at home under normal conditions of use. At least two hours after the first application, the volunteers provided a subjective assessment of the product. The results of the subjective assessment are shown in FIG. 1. The subjective assessment of FIG. 1 shows that the product was well received by the test volunteers.

It is to be understood, however, that even though numerous characteristics and advantages of the present invention have been set forth in the foregoing description, together with details of the structure and function of the invention, the disclosure is illustrative only, and changes may be made in detail, especially in matters of shape, size and arrangement of parts within the principles of the invention to the full extent indicated by the broad general meanings of the terms in which the appended claims are expressed.

1. An anhydrous fill composition for a topical product, said fill composition comprising:
(a) a branched chain hydrocarbon base oil having 9-16 carbon atoms per molecule;
(b) a sufficient amount of a viscosity adjusting agent to provide a viscosity of said anhydrous base composition of from about 10,000 cP to about 60,000 cP;
(c) one or more UV filters.

2. An anhydrous fill composition as claimed in claim 1, wherein said base oil is isododecane and said viscosity adjusting agent is a cross-linked siloxane polymer.

3. The anhydrous fill composition as claimed in claim 1, wherein said base oil is isododecane.

4. The anhydrous fill composition as claimed in claim 1, wherein said UV filter comprises an alkyl methoxy cinnamate.

5. The anhydrous fill composition as claimed in claim 1, wherein said UV filter is selected from the group consisting of ethyl hexyl methoxy cinnamate, octyl methoxy cinnamate, isoamyl methoxy cinnamate and mixtures thereof.

6. The anhydrous fill composition as claimed in claim 1, wherein said UV filter is selected from the group consisting of ethyl hexyl methoxy cinnamate, octyl methoxy cinnamate, isoamyl methoxy cinnamate, oxybenzone, homosalate, octocrylene, octyl salicylate, avobenzone, titanium dioxide, zinc oxide, diethylamino hydroxybenzoyl hexyl benzoate and mixtures thereof.

7. The anhydrous fill composition as claimed in claim 1, further comprising a photostabilizer.

8. The anhydrous fill composition as claimed in claim 7, wherein said one or more photostabilizers comprise at least one photostabilizer selected from diethylamino hydroxybenzoyl hexyl benzoate, ethylhexyl methoxycrylene, and diethylhexyl syringylidene malonate.

9. The anhydrous fill composition as claimed in claim 1, further comprising at least one UV defense booster.

10. The anhydrous fill composition as claimed in claim 9, wherein the UV defense booster is selected from photostabilizers, film-forming agents and antioxidants.

11. The anhydrous fill composition as claimed in claim 1, wherein said viscosity adjusting agent is a product of a reaction of a quaternary ammonium salt and hectorite or bentonite.

12. The anhydrous fill composition as claimed in claim 1, wherein said viscosity adjusting agent is selected from the group consisting of cross-linked siloxane polymers, polydimethylsiloxanes, and synthetic waxes.

13. The anhydrous fill composition as claimed in claim 1, wherein said viscosity adjusting agent comprises at least one silicone elastomer in combination with at least one synthetic wax.

14. The anhydrous fill composition as claimed in claim 1, wherein said viscosity adjusting agent comprises at least one silicone elastomer in combination with at least one clay-based viscosity control agent.

15. The anhydrous fill composition as claimed in claim 1, wherein said viscosity adjusting agent comprises at least one silicone elastomer in combination with at least one synthetic wax and at least one-clay based viscosity control agent.

16. The anhydrous fill composition as claimed in claim 1, wherein said viscosity adjusting agent comprises at least one silicone elastomer in combination with at least one synthetic wax, at least one clay-based viscosity control agent and at least one silica-based viscosity control agent.

17. The anhydrous fill composition as claimed in claim 1, wherein said viscosity adjusting agent is selected from the group consisting of INCI polysilicone-11, INCI vinyldimethyl/trimethylsiloxysilicate/dimethicone crosspolymer, INCI dimethicone/vinyl dimethicone crosspolymer and INCI dimethicone crosspolymer.

18. A capsule containing a fill composition as claimed in claim 1.

19. A capsule as claimed in claim 18, wherein said capsule is a soft gel capsule.

20. A capsule as claimed in claim 19, wherein said capsule is sized to provide a daily unit dose.
